(19) United States
(12) Patent Application Publication
(10) Pub. No.: US 2010/0210584 A1
(43) Pub. Date: Aug. 19, 2010

(54) LIQUID OR PASTE COMPOSITIONS INTENDED TO PROVIDE ELEMENTS ESSENTIAL FOR THE SYNTHESIS AND FORMATION OF PROTEOGLYCANS, IN PARTICULAR, FOR THE TREATMENT OF CARTILAGE DEGRADATION

(76) Inventors: Mathieu Borge, Paris (FR); Patrick Laduree, Vincennes (FR); Laurence Paris, Montmarault (FR)

Correspondence Address: JEROME D. JACKSON (JACKSON PATENT LAW OFFICE), 211 N. UNION STREET, SUITE 100, ALEXANDRIA, VA, 22314, US

(21) Appl. No.: 12/666,790

(22) PCT Filed: Jul. 4, 2008
(86) PCT No.: PCT/FR08/51250

§ 371 Date: Apr. 30, 2010

(30) Foreign Application Priority Data
Jul. 4, 2007 (FR) 0756271

(51) Int. Cl. A61K 31/715

(52) U.S. Cl. 514/54

(57) ABSTRACT

The invention relates to the field of pharmaceuticals, dietary supplements and food and, more specifically, to the field of dietary supplements intended for the treatment of degradation of cartilage of any origin. The invention relates to liquid or paste compositions based on glucosamin and chondroitin sulphate, intended to provide elements essential for the synthesis and formation of proteoglycans, in which the chondroitin sulphate/glucosamin combination is stabilised with the addition of carboxylic acids such that the pH of the medium is between 2 and 5 and the chemical degradation rate of the active substances is less than 10% when stored at 25° C. and 60% relative humidity for 10 months for doses of between 300 and 2400 mg chondroitin sulphate and between 500 and 3000 mg glucosamin. The invention is suitable for the treatment of cartilage degradation and, more specifically, arthrosis.

### TECHNICAL FIELD

This invention relates to the field of pharmaceuticals, food supplements and food and more specifically to the field of food supplements intended to treat cartilage degradation of any origin.

### DESCRIPTION OF THE PRIOR ART

Osteoarthritis is a cartilage disease. It can be primary, associated with an abnormality in the cartilage, or secondary to an abnormality of another element of the joint such as the ligaments or the menisci. It can be associated with another rheumatic disease.

Osteoarthritis is a common pathology. While the frequency increases with age, osteoarthritis can begin early in life, starting around in mid-life. It is therefore not related only to age.

It is estimated that around 10% of people over 60 years of age suffer from osteoarthritis, a condition that adversely and lastingly affects their quality of life. Intermittent complaints of pain are much more frequent. Up to 75% of the population over 65 years of age have radiological osteoarthritis affecting at least one joint. It also affects around 10% of people under 40 years of age.

Before 50 years of age, osteoarthritis is more common in men. This is undoubtedly related to the lifestyle of men, who do more manual labor and play more high-risk sports.

Early osteoarthritis is in particular related to accidents with injury to healthy cartilage. The changes caused by the accident (joint fracture, meniscectomy, taxing of the joints, loosening of the joints) result in unusual mechanical stresses that promote the destruction of cartilage and therefore osteoarthritis.

After 50 years of age, it appears that the prevalence of osteoarthritis reverses, with female predominance.

The aging of the population, overall better health conditions, and the need to maintain an active life after retirement make the preventive and curative treatment of osteoarthritis a major issue of this century.

The mechanism of osteoarthritis remains unknown. It is assumed that the imbalance between the production and destruction of cartilage begins early, and that the symptoms occur late, in favor of a particular evolution, promoting the development of a local inflammatory reaction.

This inflammatory reaction is largely responsible for painful symptoms. Cartilage is a living tissue, resulting from a balance between physiological production and destruction. Chondrocytes, cells specific to cartilage, are involved in these processes. With time, and without consideration of advanced age, an imbalance can occur in favor of destruction, as “the chondrocytes are overwhelmed in their functions”. The osteoarthritic mechanism, i.e. the degradation of cartilage, occurs and will develop fairly quickly.

Osteoarthritis is essentially a cartilage disease, resulting from excessive destruction of the extracellular matrix not compensated for by reconstruction. It is accompanied by modifications of the subchondral bone (in contact with the cartilage) and reactions of the synovial tissue of the joint affected.

The extracellular matrix of the cartilage consists of type-II collagen for 40%, type-I collagen and elastic fibers.

The extracellular matrix is present everywhere in the body, but its abundance and composition vary according to the tissue: it is very abundant in loose connective tissue, localized more specifically in bone and cartilage tissue, and very sparse in the epithelial cells. The main macromolecules of the extracellular matrix are polysaccharides (glycosaminoglycans and proteoglycans) and fibrous proteins, both structural (collagen and elastin) or adhesion-type (fibronectin and laminin), playing an important role in cell-cell and cell-extracellular matrix interactions.

The precise identification and location of the different molecules present in the extracellular matrix (essentially protein and glycoprotein) enable interactions between cells and between cells and the extracellular matrix to occur, at work in a very large number of embryological, physiological and pathological processes. For example, the chondrocytes ensure homeostasis of the cartilage by regulating the synthesis and degradation of the collagen and proteoglycans of the cartilage matrix.

The main polysaccharides of the extracellular matrix are glycosaminoglycans and proteoglycans. Glycosaminoglycans are unbranched long-chain polysaccharides formed by a repetition of the same disaccharide unit. The disaccharides of this unit comprise a monosaccharide A (glucuronic acid, iduronic acid or galactose) and a monosaccharide B (N-acetylglycosamine or N-acetylgalactosamine).

The main glycosaminoglycans present in the extracellular matrix are hyaluronic acid, chondroitin-sulfate, dermatan-sulfate, heparan-sulfate, heparin and keratan-sulfate. Hyaluronic acid is characterized by a single long chain of a several thousand sugar radicals, with the absence of sulfate groups. Numerous extracellular proteins of the extracellular matrix (collagen, fibronectin, laminin) as well as cell surface receptors (such as CD 44) can bind to hyaluronic acid. Glucosamine stimulates the synthesis of glycosaminoglycans, proteoglycans and hyaluronic acid. It is also the preferred substrate of glycosaminoglycans.

Proteoglycans are formed by a protein core to which glycosaminoglycans bind. The most widespread are decorin (chondroitin-sulfate/dermatane-sulfate) present in all connective tissues, perlecan (heparan-sulfate) in the basal membranes, and aggrecan, which is abundant in the cartilage. Hyaluronic acid does not form proteoglycans. However, proteoglycan aggregates correspond to a hyaluronic acid molecule to which multiple proteoglycans bind. Their high negative charge enables them to hold large amounts of water. Proteoglycans have the capacity to bind certain cytokines or growth factors, and thus modulate their bioavailability.

These high-molecular-weight glycosaminoglycans attract water, which explains the high water content of cartilage, a primary factor in mechanical strength.

The degradation of the extracellular matrix is due to enzyme activity, metalloproteases (MMP) and aggreganases (ADAMTS). Other proteolytic enzymes contribute to the degradation of the matrix: cathepsin B, L and K. There is also a deficit of metalloprotease inhibitors, causing the metabolic scale to lean toward catabolism (destruction). Inflammation enzymes, cytokines, in particular secreted by the synovial tissue, accentuate the degradation and favor the decline of the chondrocyte.

Osteoarthritis is responsible for painful symptoms, deformations of the joint and impaired quality of life.

At the clinical level, the functional sign of osteoarthritis is pain, mechanically induced, i.e. caused by movement and relieved by rest, and not causing nocturnal awakening.

During flare-up periods, morning stiffness may exist, but it rarely lasts longer than 30 minutes.

Pain and stiffness cause functional difficulties, which will be evaluated and monitored with standardized functional pain scales (Lequesne, Dreiser, WOMAC indices, etc.).

There may be swelling, indicating synovial effusion and deformation, sometimes visible, signs of osteophytosis, bone protuberances or off-centering of the joint.

The joints most commonly affected are: the knees, the hips, fingers and the vertebral column.

The development of osteoarthritis can be described in a number of ways:
slow and progressive over several decades;
slow and with flare-ups, over several decades, in which the synovial membrane is overwhelmed by activity, resulting in inflammation;
rapid and destructive in 1 to 2 years, especially after 65 years of age.

While osteoarthritis is a fairly slow process, the fact remains that the earlier the disease begins the more likely it is that the secondary disability will occur early. It is not rare to see 50-year-old subjects with signs of osteoarthritis.

The correction of certain factors contributing to osteoarthritis—such as excess weight, certain vitamin or hormonal deficiencies, proper treatment of joint injuries—does not necessarily provide protection from osteoarthritis.

In young subjects, there may be complete recovery. But usually, once the disease has occurred, the development, depending on various possibilities, will move toward complete loss of cartilage and destruction of the joint.

Numerous studies appear to show that osteoarthritis is more common in women, in whom it develops more quickly. Sex hormones, such as estrogen, have a favorable activity on the cartilage. Osteoarthritis of the hip is less common in women receiving hormone replacement therapy after menopause.

In some patients, a number of joints are affected, often including the fingers. This situation is very often debilitating.

While in women hormones have a favorable activity on cartilage, this is not the case for joint injuries.

Indeed, various types of injuries can lead to osteoarthritis:
direct injury to a cartilage component” direct impact, for example of the knee cap due to a fall or a blow,
injury causing a lesion of a constituent of the joint: ligaments, tendons, menisci,
less serious injuries, but repeated in certain professions and in particular in athletes with intense activity or improper form.

The description of the mechanism of osteoarthritis as well as the clinical signs identified enable various types of treatments to be provided.

Thus, the treatment of osteoarthritis is based on the improvement of symptoms, a lifestyle limiting overuse of the joint and, in some cases, the use of “corrective” techniques.

“Corrective” techniques include surgeries such as debridement, resection of affected bone areas, partial replacement of joint surfaces, prosthesis implantation, and so on.

With regard to health practices, measures are taken to limit overuse of the joints causing joint microtrauma, such as:
relative rest of the joint during flare-ups,
reduction in body weight, reduction in physical activity and non-participation certain high-risk sports,
regular physical activity; studies have shown, for example, the beneficial effect of walking for one hour, three times per week for osteoarthritis of the knee,
preserving joints by preferring actions that are more comfortable for the joint and by paying attention to joint fatigue when it occurs. It is also necessary to avoid carrying heavy loads, walking on irregular ground, and standing for prolonged periods. Do not hesitate when necessary to use a cane on the healthy side. Adopt good health practices by arranging the environment (for example, the bathroom, the kitchen, the toilets) for one's physical capabilities and by adapting one's activities according to one's physical condition.

The following is meant by improvement of symptoms:
reduction in pain,
and slowing of development.

Pain reduction is achieved by administering a certain number of drugs. Among these are:
analgesics,
non-steroidal anti-inflammatories (NSAIDs),
corticosteroids,
viscoelastic products,
lavages.

The most commonly used analgesic is paracetamol. Prescribed at effective doses, i.e. 3 to 4 g per day, it must be tried first, as indicated by the European recommendations: European League Against Rheumatism—2003 and 2005.

In the case of NSAIDs, their objective is not only to relieve pain, but also to combat inflammation that occurs during congestive osteoarthritic flare-ups.

They are generally prescribed for a short period of 10 days, but may cause digestive problems.

No type of NSAID is more effective than any other; this is dependent on the person in whom it is administered. However, no NSAID has been demonstrated as being effective in slowing the progression of osteoarthritis, even in cases of prolonged use.

The new non-steroidal anti-inflammatories called coxibs have an efficacy equivalent to the other NSAIDs, but appear to have better digestive tolerance.

Anti-inflammatory gels and pomades are sometimes used locally for superficial joints such as fingers and knees. However, their use can only be prescribed by a physician because these products, depending on their components, may trigger irritation or cause reactions when exposed to sun.

Corticosteroids are intended to fight inflammation locally. They are administrated as injections directly into the joint. They can be very useful for getting through a difficult period during an osteoarthritic flare-up that is not successfully controlled by the oral drug treatments. Their efficacy generally lasts for one to two months.

However, excessive injections (more than three per year for the same joint) of corticosteroids are not without risk: joint impairment over the long term and risk of infection.

The fourth category of products used to reduce the problem of pain in osteoarthritis is represented by products having a certain viscosity.

Thus, the injection of hyaluronic acid into the joint involves replacing the synovial liquid of the arthritic knee joint with a gel of which the properties are the same as those of a healthy joint liquid.

For a given joint, three to five injections are performed with one-week intervals.

Efficacy is determined by a decrease in joint pain and an improvement in the functional state of the joint. It takes longer than corticosteroids, but lasts longer (8-9 months on average).

Unlike corticosteroid injections, these are not a treatment for osteoarthritic flare-ups.

More than a category of products, one mode of treatment enables pain to be reduced: joint lavages.

This is a technique that is applied to the knee joint and that is intended to remove impurities (cartilage fragments) from the joint. Performed with physiological serum, this is a procedure that requires local anesthesia; it lasts for 30 to 60 minutes and must be performed in a hospital. It is systematically followed by a corticosteroid injection. Its efficacy may last from 6 to 12 months.

However, aside from surgical treatments, no other treatment cited above is intended to stop or slow the development of the disease, i.e. degradation of cartilage.

One category of products has this capability: SYSADOA (Symptomatic Slow-Acting Drugs for Osteoarthritis).

They have been shown to be effective for symptoms (pain, stiffness and joint swelling), and, for some, a capacity to activate the production of cartilage constituents.

At present, only four products potentially have this activity:
diacererin
soy unsaponifiables
chondroitin sulfate
glucosamine

These products are characterized by an effect that is:
persistent on cartilage: their efficacy lasts several months after the treatment has been stopped;
delayed on symptoms: between 15 days (Qiu) and 8 weeks.

Their tolerance is good to excellent.

Diacerein is a moderate anti-inflammatory that stimulates the production of proteoglycans, glycosaminoglycans and hyaluronic acid.

Clinical placebo-controlled studies have shown a significant decrease in the articular joint space, which represents the thickness of the cartilage at the joint.

However, this product is provided only with medical prescriptions, and is not available over-the-counter.

In the case of soy unsaponifiables, clinical studies have shown that they lead to a reduction in the use of anti-inflammatories. This effect extends even when the unsaponifiables are no longer being taken. However, the effect on the decrease in the articular joint space is not significant.

Chondroitin sulfate is a natural constituent of cartilage. It is capable of binding to the cartilage after oral administration and also stimulates the production of proteoglycans by the chondrocyte.

In animals, it has been demonstrated that labeled chondroitin was absorbed at more than 66% and was found in the synovial liquid and cartilage.

In humans, absorption is lower, at around 15%, but is dependent on the molecular weight.

At the clinical level, the analysis of specific published data (randomized, double-blind, multi-center study) shows that chondroitin administered at a dose of 800 to 1200 mg/day:
improves the Lequesne index (scale measuring pain and stiffness causing functional impairment),
improves mobility,
reduces pain, and
preserves the articular joint space.

In France, like abroad, this molecule is:
a product available by prescription, such as Chondrosulf®, Structum®,
but is also sold over-the-counter in the form of food supplements under the name “chondroitin sulfate” or under fanciful names.

In both areas, the doses used vary from 400 mg to 1200 mg per dose and are available in different forms: capsules or tablets.

A retrospective study in the field of chondroitin-based galenic forms did not identify a patent relating to this molecule administered orally at doses between 400 mg and 1200 mg and even greater, regardless of the form: solid, liquid or pasty.

A single patent mentions the use of chondroitin sulfate in liquid form, but for ocular application: patent EP 0063973.

Like chondroitin sulfate, glucosamine is a natural constituent of cartilage. It is included in the cells of the extracellular matrix formed by glycosaminoglycans and proteoglycans.

This molecule is intended to stimulate the synthesis of glycosaminoglycans, proteoglycans and hyaluronic acid.

In the treatment of osteoarthritis, the administration of glucosamine is intended to stimulate the production of the elements cited above, which become defective in this disease.

When taken orally, the absorption of glucosamine is on the order of 90%, and its maximum concentration is reached in 8 hours.

Identical to chondroitin, glucosamine is found preferentially after administration in the joint cartilage and the bone.

At the clinical level, double-blind studies have shown that glucosamine was more effective than ibuprofen on joint pain and swelling, beginning in the second week of treatment.

The same observation is made with regard to paracetamol, but over the long-term, after six months.

Similarly, glucosamine preserves the articular joint space, thus delaying the need to use “corrective” techniques.

The active doses in this area are on the order of 1500 mg/day.

Unlike chondroitin, glucosamine is not a prescription molecule, but is used in the composition of many food supplements.

In most cases, these are liquid forms of which the glucosamine concentrations vary from 30 mg to 1500 mg per dose, such as:
Artol®: 30 mg of glucosamine per ampoule
Artrofluide®: 1500 mg of glucosamine per ampoule
Matol Glucosamine®: 500 mg.

Numerous patents have been issued in this field regarding the use of glucosamine alone as well as in combination with other components aside from chondroitin sulfate.

Among these patents, we can cite:
patents implementing glucosamine salts:
   patent FR 9710326, crystallized glucosamine sulfate
patent CA 2573741, N-acetyl-glucosamine
   patent US 2007 048354, use of N-acetyl-glucosamine in infant preparations and formula milk
patents protecting pharmaceutical forms:
   gels, patent US 2007 048386
   creams, patent CA 2493947
   tablets, patent JP 2005 225782 extended-release form, patent WO 2005 087202
      patents protecting the incorporation of glucosamine in food preparations or drinks:
   in beer, patent, WO 2004 085603
   in milk, patent WO 2004 093556
   in wine, patent WO 2004 085604
   in drinks, patent US WO 2004 071855
      patents protecting combinations of glucosamine/other active principles, other than chondroitin sulfate:
   glucosamine/pinitol, patent KR 2007 0002401
      glucosamine/non-steroidal anti-inflammatories: patent WO 2005 116086, patent US 2006 280811
   glucosamine/diclofenac, patent RU 2292201
   glucosamine/vitamin B1, patent KR 2002 0036752
   glucosamine/theaflavin, patent WO 2006 128032
   glucosamine/vitamin D, patent BE 1015783.

Even if glucosamine has a certain efficacy associated with other molecules such as anti-inflammatories, one of the best combinations intended to provide essential elements for the synthesis and constitution of proteoglycans in order to fight cartilage degradation remains the combination of glucosamine and chondroitin sulfate.

Indeed, a recent study conducted by Clegg D O et al over a six-month period on 1583 patients with gonarthrosis demonstrated that, for doses of 1500 mg/day of glucosamine and 1200 mg/day of chondroitin, the treated group was 79.2% more receptive (pain reduction) than the placebo group (54.3%).

The combination of glucosamine/chondroitin sulfate has been the subject of a number of patents:
for use in osteoarthritis itself or for other uses. As other uses, we can cite:
   the Korean patent KR 2004 0100009, protecting an ultrasonography gel of which the concentration of chondroitin or glucosamine alone and in combination varies from 0.01 to 10%,
   the American patent US 2002 34978 claiming the use of this combination in the urinary field, in which this combination is in the form of a dissolvable powder,
in combination with at least one third active ingredient such as:
   in the presence of an analgesic, patent WO 2007 001708, protecting a tablet form,
   in the presence of chitosan and plants, Korean patent KR 2004 0004346
   in the presence of plants:
      American patent US 2004 161480
      Safflower oil, Korean patent 2001 0046562, protecting a solid form or a solution
      Scutelleria, American patent US 2006 165821, protecting a tablet form
      Boswellia (active anti-cancer plant) American patent US 2006 040000 protecting an oral form
      ginseng, American patent U.S. Pat. No. 6,979,458, protecting a suspension
      Kernel oil, American patent US 2004 180100, protecting a solid divisible form
   in the presence of trehalose, a sugar improving the flavor of the preparation and facilitating the therapeutic activity of the combination, patent EP 1354590
   in the presence of hyaluronic acid:
      for ocular use, patent WO 2006 058109
         for veterinary use, American patent US 2005 182022
      sterile application, patent WO 2004 034980
   in combination with hydroxycitric acid, American patent US 2005 282772
   in combination with calcium, American patent U.S. Pat. No. 6,969,533
   in combination with other elements, German patent DE 202005006527
   in combination with sulfide, patent WO 2005 041999
   in combination with the “COX 2 inhibitor” American patent US 2005 101563
   in combination with magnesium and carotene, American patent US 2004 254142 protecting a solution.

Whether it is in the form of a simple combination of chondroitin sulfate/glucosamine or in the form of a complex combination, most of the patents also protect the galenic form administrable to humans or animals:
solid galenic form such as capsules, effervescent or non-effervescent tablets with or without delayed release, powders, and so on. We can add the following to those cited above:
   the Korean patents KR 2003 0092698, KR 2003 0013836 protecting the combination of chondroitin sulfate/glucosamine in capsule form, of which the percentage of active principles varies from 0.3% to 1.85% chondroitin and 0.5% and 3.7% glucosamine,
   patents WO 2005 079764 and US 2004 234599, protecting delayed forms of glucosamine/chondroitin sulfate of which the active principles are incorporated into pellets or into a matrix,
   the Japanese patent JP 2004 002482, protecting a solid form of chondroitin sulfate/glucosamine. The American patent US 2003 134825 protecting an effervescent chondroitin sulfate/glucosamine tablet,
   etc.
pasty galenic form such as gels, creams, etc. In addition to the patents for complex combinations of chondroitin sulfate/glucosamine are:
   the Russian patent RU 2260432 and the American patent US 2003 045503 relating to a topical form in an aqueous medium and in an anhydrous medium,
   the American patent US 2005 232980 relating to a transdermal form,
   the patent WO 2004 012665 and the American patent US 2003 212005 relating to a gel for human or animal administration,
   the American patent US 2003 134825 relating to the introduction of glucosamine and chondroitin sulfate in a pudding,
   etc.
liquid galenic form such as drinkable solutions and suspensions:
   the American patent U.S. Pat. No. 6,979,458 protecting a glucosamine/chondroitin sulfate suspension,
etc.

However, the various patents cited above, whether for glucosamine or chondroitin used separately or in a simple or complex combination with other ingredients do not mention stability studies on the end product. It is known to a person skilled in the art that the development of drugs or food supplements, or even foods, requires stability studies in order to demonstrate incompatibilities between the various ingredients, the active principles with one another or with the carriers.

By “ingredients”, we mean all substances, active or not, used in the composition of an end product, whether it is liquid, solid or pasty.

By “active principles”, we mean all substances having a beneficial effect on the body after administration.

By “carriers”, we mean all substances that contribute to the production of any galenic form without having a real activity on the human or animal body.

Thus, it has long been known that sugars in a liquid medium and, to a lesser degree, in a solid medium, react over time and under heat with all components containing nitrogen atoms (amino derivatives). A complexation reaction occurs progressively between the carboxyl (HCO) and even hydroxyl (HO) groups and the nitrogen (N) atoms resulting in a brown coloring of the medium. This reaction is more or less intense according to the reactivity of the nitrogen atom. In the pharmaceutical medium, this reaction is known as a Maillard reaction, causing a more or less brown coloring of syrups under heat and a more or less beige coloring of tablets.

This reaction can also occur when sugars are chained together to form polysaccharide polymers such as chondroitin. This reaction is even more likely if the polymer is combined with a sugar such as glucosamine: amino sugar.

Thus, after an in-depth study of the literature in the field of simple or complex glucosamine/chondroitin combinations presented in liquid form, it has not been possible to identify an effective solution for stopping or slowing the appearance of the Maillard reaction.

### DESCRIPTION OF THE PATENT

In view of this situation, and in order to overcome it, the invention proposes liquid or pasty compositions based on glucosamine and chondroitin sulfate intended to provide essential elements for the synthesis and constitution of proteoglycans, in which the combination of chondroitin sulfate and glucosamine is stabilized by the addition of carboxylic acids resulting in a pH of the medium of between 2.0 and 5.0 and a chemical degradation of the active substances below 10% at 25° C. and 60% relative humidity after 10 months, for doses of between 300 and 2400 mg for chondroitin sulfate and between 500 and 3000 mg for glucosamine.

Thus, the present invention is intended to develop liquid or pasty compositions of which the pH is such that it enables chemical stability to be achieved in the combination of glucosamine and chondroitin sulfate over a commercially-acceptable time period.

By “liquid compositions”, we mean all preparations having a liquid phase of which the percentage by weight with respect to the total weight of the composition is greater than or equal to 50%, in which said liquid phase can contain particles in suspension.

By “pasty compositions”, we mean all liquid preparations having a viscosity greater than or equal to 100 Centipoises (cPs), such as gels, creams and so on.

This invention is also applicable to compositions intended to provide essential elements for the synthesis and constitution of proteoglycans in order first to slow cartilage degradation systemically (orally) or locally (topically). These compositions can also contain:
other active principles in order to amplify the anti-inflammatory action of these compositions
and other carriers in order to improve the taste of the product (oral form) or the transdermal passage for the topical form.

This invention is based on the fact that certain substances in an acid medium act as weak acids. This is the case for amino derivatives, which are essentially basic products. However, in an acid medium, the amino group, inter alia, nitrogen, is ionized by the appearance of a positive charge characteristic of acid functions. This positive charge is neutralized by the presence of anions (negative charge) in the medium thus preventing the complexation reaction between the free nitrogen group and the carboxyl functions and, to a lesser degree, the hydroxyl functions of the sugars or polysaccharides solubilized in the carrier of the liquid or pasty composition.

Therefore, the liquid or pasty compositions thus obtained become more stable over time, which is physically manifested by a disappearance or a slow appearance of the brown coloring characterizing the complexation reaction between the free amino groups and the hydroxyl groups also called the Maillard reaction.

The acidifying substances or substance maintaining a pH of between 2.0 and 5.0 are classically used in the pharmaceutical, cosmetic or dietetic fields.

### DETAILED DESCRIPTION

In general, the Maillard reaction is a chemical process that appears in most products containing sugars and amino derivatives.

By “sugars”, we mean all components having hydroxyl and carboxyl functions on the skeleton of the molecule, whether it is simple such as glucose, fructose, etc., or complex such as polysaccharides. All of these compounds belong to the carbohydrate class.

It is a highly complex reaction that is initiated by a reaction between the nitrogen group of the amino substance and the carboxyl function of the sugars by delocalization of the free electrons present on the molecules (movement of the electrons) thus promoting attraction between atoms.

At the reaction level, the initial step of the Maillard reaction occurs as follows:

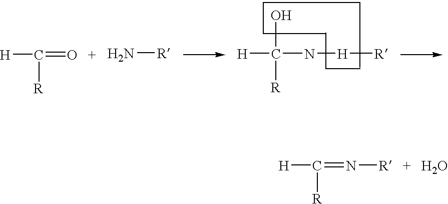

This reaction is therefore an electronic rearrangement at the level of the carboxyl function of the sugar and the nitrogen atom of the amino derivative: the nitrogen atom captures the electrons of the carbon from the sugar by delocalization of the electrons of the C═O double bond. The nitrogen atom is then ionized, enabling the sugar to bind to the amino derivative. Water is then eliminated, resulting in a new compound.

The sugars are generally represented by RCHO, a carboxyl function (CHO) and the substance aminated by H₂NR′, an amine function (NH₂).

After this first step, ionization and then isomerization reactions occur, promoted by the aqueous medium in which the various molecules are located. Finally, cyclization reactions take place, resulting in colored derivatives.

The Maillard reaction therefore occurs by a mechanism of condensation between an amino derivative and a sugar in an aqueous medium.

By “condensation”, we mean any chemical mechanism that enables one molecule to bind to another.

This condensation occurs progressively over time and under heat.

The greater the heat is, the more advanced the degradation will be, resulting in products colored by cyclization due to dehydration of the molecules (loss of a water molecule).

Thus, according to the molecules present in the medium and the storage conditions, a yellow coloring slowly occurs until a caramel-brown solution is obtained.

This reaction is less intense when the sugar is a polyol or a polysaccharide.

This is due to the fact that the chaining of the sugar molecules causes steric hindrance thus limiting interaction with the aminated molecules.

At the chemical level, glucosamine, which can be of either natural or synthetic origin, is a glucose molecule to which an amino grouping has bonded. Consequently, in water without the addition of sugar, this molecule automatically leads to the appearance of a brown color of the solution by the condensation reaction of the different glucosamine molecules together. This appears clearly in an aqueous 7.5% glucosamine solution. After 8 months at 40° C., the solution takes on a caramel hue.

To prevent the appearance of coloring over time in glucosamine solutions, preference is given to a “prodrug”, N-acetyl-glucosamine.

By “prodrug”, we mean stable chemical combinations that release the active substance in non-degraded form at the required dose in the body.

In the case of N-acetyl-glucosamine, its administration leads to the release of glucosamine and acetic acid, of which only glucosamine has a therapeutic activity.

N-acetyl-glucosamine has the following formula:

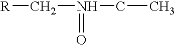

The migration of a hydrogen of the NH group to the sugar molecule as described above thus becomes impossible in the case of N-acetyl-glucosamine: two hydrogen atoms are needed to trigger the Maillard reaction.

In the patent KR 2001 0029674, owing to the greater heat stability of N-acetyl-glucosamine, it is used instead of glucosamine in order to obtain a stable composition in the presence of chondroitin sulfate.

In this invention, glucosamine is combined with chondroitin sulfate, which has the special property of being a polysaccharide polymer.

This molecule consists of a chain of molecules of N-acetyl-glucosamine and glucuronic acid, which is simply an acid carbohydrate.

When placed in solution in water in an amount of 6.0% with respect to the total weight of the composition, the solution thus obtained has a coloring that is ten times less intense than that of a pure 7.5% glucosamine solution. This demonstrates the essential action of the acetyl group on the stability of the glucosamine molecule in the presence of other carbohydrates such as glucuronic acid.

Many patents mention the combination of these two molecules: glucosamine and chondroitin sulfate in an aqueous and pasty medium. However, none refer directly or indirectly to chemical stabilization by any means, of such a combination.

Aside from blocking the amine function of glucosamine by an acetyl group or the like, resulting in a new molecule or “prodrug”, it is possible to stabilize the NH₂ group by a simpler reaction that takes place at the time of production of the solution.

Indeed, this blocking of the amine function according to the present invention is based on the fact that the NH₂ group in an acid medium undergoes protonation (appearance of positive charges) and enables the binding of an anion (negative charge) preventing delocalization of the hydrogens during initialization of the Maillard reaction described above.

This chemical reaction implements carboxylic acids such as citric or oxalic acid, etc. It has the following equations:

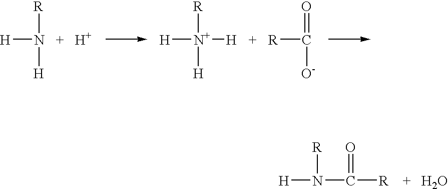

Acid medium->protonation->blocking and elimination of water

This reaction takes place under cold or hot conditions.

This blocking has been demonstrated with solutions of chondroitin sulfate and glucosamine with or without the presence of citric acid.

Three solutions have been produced, each containing 7.5% glucosamine by weight with respect to the total weight of the composition and 6.0% chondroitin sulfate by weight with respect to the total weight of the composition. In two solutions, anhydrous citric acid was added before the introduction of chondroitin sulfate. One of the solutions was heated at 100° C. for 10 minutes.

After 8 hours of storage at 40° C., the solutions containing citric acid remained colorless, and the solution without citric acid took on a caramel color.

This experiment showed that:
the Maillard reaction is almost instantaneous between glucosamine and chondroitin sulfate, because the latter has a very high reactivity owing to the free sulfate function SO₃²⁻. The delocalization of the electrons of this group is very significant, thus promoting the Maillard reaction;
the citrate group (negative charge) clearly binds to the protonated amino function of the glucosamine according to the reaction described above, thus blocking the Maillard reaction.

However, among all of the patents cited above, none mentions chemical stabilization of the glucosamine/chondroitin sulfate solution in an acid medium, in order to block the amino function so as to slow the Maillard reaction. In some cases, such as in patents KR 2004 0100009 and U.S. Pat. No. 6,730,331, the gel developed has an alkaline pH making the combination even more unstable because, aside from the Maillard reaction, the glucosamine degrades rapidly beyond a pH of 7.0.

Similarly, the patents WO 2006 058105 and GB 1091535 protect an ocular preparation with a neutral pH, which is necessary for such an application. The preparation must be preserved in cold conditions before use.

The patent US 2004 254142 mentions the use of citric acid in order to counteract the pH of the medium, but without indicating the pH range, or the fact that it is used to stabilize the solution.

The patent US 2003 134825 as well as patent US 2003 138543 protect the use of citric acid as a flavor enhancer and buffer solutions for modifying the melting point of the gelling agents used to produce puddings and other pastries. No reference is made to maintaining the chemical stability of the glucosamine/chondroitin sulfate combination.

In general, citric acid is widely used in the pharmaceutical, dietetics and food industries for liquid forms as a flavor enhancer, owing either to the acidity that it gives preparations or to the flavor intensification. Thus, in many patents protecting the combination of glucosamine/chondroitin sulfate combined with other active principles, citric acid is cited for its role at the gustatory level and not as an agent for chemical stabilization of the preparation. We can thus cite, in this case, the following patents:
KR 2001 0018321
WO 2004 004686
WO 2004 002423
US 2033152642
JP 2001 39408
WO 2004 004686, WO 2004 002423, US 2003 152642.
   These patents protect a process of production of dietetic bars as well as sports drinks. Citric acid is used as a flavor enhancer and to adjust the pH of the drinks in the case of effervescent drinks. No mention is made of the addition of citric acid as a chemical stabilizer of the preparation.
IE 981006. This patent protects the incorporation of citric acid in a powder in order to produce a drinkable solution. No reference is made to the use of citric acid as a chemical stabilizer for the glucosamine/chondroitin combination.

Other patents protect the chondroitin sulfate/glucosamine combination for other applications, such as:
transdermal systems, patent US 2005 232980. Citric acid is used to adjust the viscosity of the transdermal preparation and not to chemically stabilize the combination.
parenteral injections, patents CA 2446615 and U.S. Pat. No. 6,476,005. These patents protect the use of malic acid as a detoxifier and citric acid in a buffer solution with sodium chloride in order to adjust the pH of the preparation. However, any person skilled in the art knows that the pH of a parenteral solution must be as close as possible to the pH of the body, i.e. 7.0. Consequently, at this value, the glucosamine/chondroitin sulfate combination is not chemically stable.

Similarly, patents US 2005 282778 and US 2003 229049 protect parenteral solutions for injection in the spinal disks. Again, the pH of such solutions is on the order of 7.0.
Solutions or creams to be applied to open wounds.
   As above, the citric acid and the monosodium and disodium phosphates are used to buffer the medium so as to have a pH compatible with the biological constants of the body, i.e. a pH of 7.0.

Patents EP 1354590 and TW 235660B protect the glucosamine/trehalose (sugar) combination and mention the instability of glucosamine in the presence of sugars. They recommend the use of acids in order to stabilize the medium without setting the pH range, and they tend to prefer a derivative of ascorbic acid. However, this patent does not protect the glucosamine/chondroitin sulfate combination of which the chemical instability is clearly greater than that of glucosamine alone.

Thus, to obtain liquid or pasty compositions according to the present invention would require substances that confer a pH of between 2.0 and 5.0 on the medium.

These substances are carboxylic acids.

By “carboxylic acid”, we mean all substances having at least one COOH acid function releasing a hydrogen (H⁺) in the medium.

These carboxylic acids can be aliphatic (linear) or aromatic (cyclic).

Thus, the invention includes acetic acid and derivatives thereof, adipic acid, azelaic acid, butyric acid and derivatives thereof, citramalic acid, citric acid and derivatives thereof, decanoic acid, diglycolic acid, dodecanedioic acid, trans-2-dodecene 1,12-dioic acid, formic acid, fumaric acid, gluconic acid, glutaric acid and derivatives thereof, glycolic acid, glyoxylic acid, hexadecanedioic acid, hexadiene-2,4-dioic acid, hexanoic acid and derivatives thereof, trans-hexene-3-dioic acid, lactic acid, lauric acid, levulinic acid, linoleic acid, maleic acid, malic acid, malonic acid, mellitic acid and derivatives thereof, methyl-3-crotonic acid, methyl-3-glutyric acid, mucic acid, myristic acid, octanoic acid, oenanthic acid, oleic acid, oxalic acid and derivatives thereof, palmitic acid, pelargonic acid, pentadecanoic acid, trans-pentenoic acid, pentynoic acid, pimelic acid, pivalic acid, propionic acid, propyl-2-valerianic acid, pyruvic acid, sebacic acid, sorbic acid, stearic acid, suberic acid, succinic acid and derivatives thereof, tartric acid, tetradecanedioic acid, tiglic acid, tridecanoic acid, undecylenic acid, valeric acid and derivatives thereof.

By “derivatives”, we mean all molecules having the same chemical radical, such as butyric acid, isobutyric acid, etc.

These substances can be used alone or in combination so as to have a pH of the medium between 2.0 and 5.0.

The inhibition of the Maillard reaction between glucosamine and chondroitin sulfate is performed in a liquid medium. This reaction is facilitated by the fact that glucosamine is a molecule that is highly soluble in water and in an alcohol medium. Depending on the nature of the acid(s) used, the solvents that may be used are either water or alcohol or a mixture of the two.

The alcohols capable of being used for this inhibition are ethanol, propanol-1 and isopropanol.

The amount of alcohol capable of being used is limited by the presence of chondroitin sulfate, which is insoluble in these solvents. Thus, the amount of alcohol capable of being incorporated into the medium is between 0.5% and 75% by volume with respect to the total volume of the liquid phase implemented for the liquid or pasty compositions.

The amount of acid capable of being incorporated into the medium is dependent on the type(s) of acid(s) implemented and in particular the number of COOH groups capable of releasing H⁺ ions.

This amount is determined by the ratio characterizing the number of acid moles used to block 1 mole of glucosamine. This ratio varies from 1:0.05 to 1:1.5 and preferably from 1:0.25 to 1:1, according to whether the inhibition reaction uses one or more acids.

Over time, according to the other ingredients present in the medium, the pH of the medium may evolve toward basic values capable of causing destabilization of the complex formed. To prevent a change in the pH of the medium, it is possible to introduce buffer solutions.

By “buffer solution”, we mean a solution that is capable of absorbing acid or basic pH variations according to their compositions, so as to maintain the pH of the medium at between 2.0 and 5.0.

Among the acid buffer solutions based on acid/salt or acid/base combinations or salt alone, the following compositions are included:
compositions based on hydrochloric acid and:
   sodium chloride
   potassium phthalate
   glycine and sodium chloride
   potassium chloride
   sodium citrate
   boric acid neutralized by sodium hydroxide (borax)
   monopotassium phosphate
   ammonium acetate
compositions based on citric acid and:
   sodium citrate
   sodium hydroxide
   disodium phosphate
compositions based on phosphoric acid and:
   sodium hydroxide
   monosodium phosphate
compositions based on acetic acid and:
   sodium acetate
   ammoniac
   sodium acetate and ammonium acetate
compositions based on succinic acid combined with sodium hydroxide
compositions based on lactic acid combined with lactate
compositions based on monopotassium phosphate and dipotassium phosphate.

The proportion of these various components enables an acid pH of between 2.0 and 5.0 to be maintained.

The inhibition of the Maillard reaction is independent of the origin of the glucosamine and the chondroitin sulfate.

Thus, the glucosamine of the present invention can be obtained either by chemical synthesis from glucose or by extraction from different substrates of animal origin.

In the case of an animal source, the glucosamine is extracted by hydrolysis of the chitin obtained from crustacean carapace. Other marine sources can be used, such as sea cucumbers and certain types of mussels. This extraction by hydrolysis can also be performed using other compounds such as amino polysaccharide polymers such as glycoproteins and glycosaminoglycans.

Depending on the mode and the source of extraction, two types of glucosamine are obtained, form α and form β. These two forms are differentiated by their physical properties.

Form α has a melting point of 88° C., a rotation of +100° and is in crystalline powder form.

Form β has a melting point of 110° C., a rotation of +28° and is in needle form.

The glucosamine, α or β, of the present invention is in the form of salts of hydrochloric acid, glucosamine hydrochloride or sulfuric acid salts, or glucosamine sulfate.

With regard to chondroitin, the only possible source is extraction from a substrate of animal origin. Owing to its polysaccharide nature, it cannot be obtained by synthesis. The substrates used for extraction consist of bovine (trachea), porcine (skin, bone, etc.) and fish (shark, skate, etc.) cartilage.

The extraction protocol consists of a proteolytic treatment of these various tissues, followed by a separation and purification process. The extraction processes enable a polymer to be obtained with an average molecular weight on the order of 50,000. Some processes result in lower molecular weights enabling better assimilation of the product.

The chondroitin sulfates used in this invention have a molecular weight capable of ranging from 10,000 to 60,000.

Chondroitin sulfate, as its name indicates, has sulfate groups on the “N-acetyl-glucosamine” unit.

Depending on the position of these sulfate groups, five types of chondroitin sulfate exist:
chondroitin 4-sulfate: chondroitin sulfate A, where R₁═SO₃H and R₂═H
chondroitin 6-sulfate: chondroitin sulfate C, where R₁═H and R₂═SO₃H
chondroitin sulfate B, where R₁═SO₃H and R₂═H with a reverse attachment at C_{5′}
chondroitin 4,6-sulfate: chondroitin sulfate E where R₁═SO₃H and R₂═SO₃H
chondroitin 2,6-sulfate: chondroitin sulfate D where R₁═H and R₂═SO₃H and a SO₃H group at C₂ of the glucuronic acid.

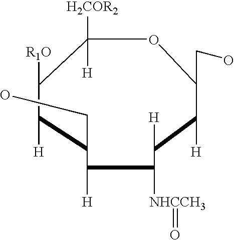

The three main forms are A, B and C, which are differentiated by their rotation:
form A: between −28° and −32°
form C: between −12° and −18°
form B: between −60° and −70°.

According to the bibliographic data regarding the use of the glucosamine-chondroitin combination in the field of joints, this invention enables protection of liquid compositions for oral and topical administration in which the glucosamine concentration is between 0.10% and 15% and that of chondroitin sulfate is between 0.06% and 12% in a single dose, by weight with respect to the final weight of the composition.

By “single dose”, we mean the packaging that enables the effective amount of active principle to be administered once per day.

These same compositions can be in multi-dose form, in which the glucosamine concentrations may vary from 2.0% to 20.0% by weight with respect to the total weight of the composition, and in which the chondroitin sulfate concentration may vary from 1.0% to 15% by weight with respect to the total weight of the composition.

By “multi-dose”, we mean liquid preparations intended to be administered a plurality of times over periods of 15 to 30 days, such as syrups.

For oral administration, a certain number of ingredients are added in order to make the composition palatable and to ensure that the microbiological cleanliness is preserved.

At the gustatory level, various categories of carriers are used:
sweeteners,
flavor enhancers,
flavors,
and, to a lesser degree, coloring agents.

The inhibition of the Maillard reaction between glucosamine and chondroitin sulfate enables classic “sugar”-type sweeteners to be introduced into the composition, because all of the amino functions of the active molecules are blocked.

Thus, this invention protects three categories of sweeteners:
natural sweeteners such as saccharose, fructose, glucose, galactose, etc.
sweeteners of natural origin: sorbitol, maltitol, xylitol, glycerol, etc.
synthetic sweeteners: aspartame, sucralose, saccharine and sodium salts, acesulfame and salts thereof.

The concentrations of sweeteners that are natural or of natural origin are between 5.0% and 50% by weight with respect to the total weight of the composition.

In the case of synthetic sweeteners, the concentrations capable of being used vary from 0.05% to 2.00% by weight with respect to the total weight of the composition.

As a general rule, any liquid composition intended for oral administration requires the flavor to be enhanced. Certain substances enable this objective to be achieved. These are flavor enhancers. In most cases, they are acids. This invention includes citric acid, malic acid, oxalic acid, tartric acid and ascorbic acid, which may or may not be combined with their salts.

The concentrations used vary from 0.1% to 10.0% by weight with respect to the total weight of the composition, according to the type of acid used and the desired flavor, acidic or not.

All liquid compositions intended for oral administration require the incorporation of a flavor. These flavors can be natural or synthetic. The concentrations used vary according to the type of flavor and the desired flavor intensity. They range from 0.1% to 10% by weight with respect to the total weight of the composition.

In some cases, the stabilized glucosamine/chondroitin combination can be flavored by a “fruit juice” base. Under these conditions, the concentration of fruit juice may vary from 20.0% to 85% by weight with respect to the total weight of the composition.

By “fruit juice” base, we mean any liquid composition of which the proportion of pure fruit extract is greater than 10%.

To facilitate administration of a liquid form, the appearance of the product, inter alia, the coloring, plays a critical role. Coloring agents can therefore be added to said liquid compositions.

These coloring agents can be natural or synthetic, and their concentration varies, according to the desired appearance, from 0.01% to 10% by weight with respect to the total weight of the composition.

In the case of liquid or pasty compositions, the introduction of preservatives is essential in order to ensure microbiological cleanliness and to maintain the latter over time. This invention includes benzoic acid and salts thereof, sorbic acid and salts thereof, methyl, propyl and ethyl parahydroxybenzoates and salts thereof.

For pasty forms for topical use, benzyl alcohol, butylhydroxyanisol, butylhydroxytoluene and propyl, octyl and dodecyl gallate can also be used.

The concentrations used vary according to the results of the “Challenge test” and are between 0.05% and 1.0% by weight with respect to the total weight of the composition.

By “challenge test”, we mean a test that enables an estimation, on the basis of a predefined dose of preservative, of the evolution or non-evolution of microorganisms incorporated into a liquid composition.

The liquid or pasty compositions of this invention can contain other active principles such as anti-inflammatories, plants, minerals, vitamins, organic sulfur derivatives, etc.

These products can be solubilized in the solvent of said compositions or placed in suspension or dispersed in the form of an oil phase in the aqueous phase containing the glucosamine/chondroitin sulfate combination.

In the case of suspensions, the particle size of the active substances other than chondroitin sulfate and glucosamine is such that no sedimentation should be observed.

The particle size of these active substances may vary from one micron (micronized form) to 500 μm.

The suspension treatment of these particles can be promoted by incorporating a suspension agent in the medium.

By “suspension agent”, we mean all substances that confer a certain viscosity on the medium, facilitating the homogeneous distribution of particles in the composition.

The suspension agents include suspension agents of natural origin such as cellulose and derivatives thereof, starches and modified starches and derivatives thereof, guar gum, xanthan gum and carrageenans.

The concentration of these different substances varies according to their nature and the desired viscosity. For pasty oral compositions, the viscosity varies from 100 cPs to 20,000 cPs. For pasty compositions intended for topical administration, the viscosity may vary from 5,000 cPs to 100,000 cPs.

The concentrations of suspension agent under such conditions range from 0.1% to 5%.

When active principles other than glucosamine and chondroitin are not soluble in the aqueous or alcohol phase, they can be solubilized in an oil or an organic solvent. In this case, the solubilized active principle is dispersed in the aqueous phase and results in the formation of a more or less viscous liquid composition called an emulsion. This emulsion is a “water-in-oil” emulsion when the oil phase is greater in volume than the aqueous phase, and an “oil-in-water” emulsion when the reverse is observed.

Such compositions require the use of oily or organic carriers in order to dissolve certain active principles.

By “carriers”, we mean all liquid substances that enable the active principles dissolved in the latter to be incorporated into the aqueous phase in solubilized form.

In the present invention, these carriers include:
vegetable oils, hydrogenated vegetable oils, ethoxylated vegetable oils: olive oil, hazelnut oil, coconut oil, castor oil, soybean oil, sesame oil, etc.
mineral oils: paraffin, isoparaffin, cycloparaffin oils, silicone oils, isohexadecane, isododecane, and derivatives, etc.
natural oils, squalane, hexamethyltetracosane, mono-, di- and triglycerides, etc.
synthetic oils: polyisobutene, hydrogenated polyisobutene, etc.
and other classic non-toxic lipophilic, hydrophilic and hydro-lipophilic solvents used to produce drug forms: polypropylene, propylene carbonate, dimethyl isosorbide ether, polyoxyethylene glycols (Macrogols), polyethylene fatty acid esters, propylene glycol fatty acid esters, propylene glycol dicaprylate/dicaprate, glycerol caprylate/caprate, polyoxyethylene/polyoxypropylene glycol fatty acid esters, triacetine, isopropyl myristate, glycofurol, liquid fatty acid esters, ethyl acetate, butanol, propylene glycol acetate, butyl acetate, ethyleneglycol monobutyl ether, ethyl lactate, butyl acetate, diethyleneglycol monoethylic ether, glycerin monooleate, glycerin linoleate, fatty acid and glycerol esters, glycerol and PEG fatty acid esters, etc.

The proportion of these different carriers is dependent on the solubility of the active principles and may vary from 1% to 75% by volume with respect to the total volume of the liquid composition.

In some cases, these solvents require the use of surfactants in order to prevent any phase shifts between the two phases.

The surfactants capable of being used in this invention are:
non-ionic surfactants:
   sorbitane esters: polysorbates, spans, tweens, etc.;
   polyethoxylated fatty acids: PEG-8 stearate to PEG-100 stearate;
   polyethoxylated fatty alcohols: mixture of PEG monolaurate ether having 4 to 23 oxyethylene groups on the polyoxyethylene chain, etc.;
   glycol esters: methylglycol stearate;
   glycerol esters: glycerol monostearate, PEG-75 stearate, glycol and PEG 6-32 stearate, etc.;
   PEG esters;
   saccharose esters;
   fatty alcohol and PEG esters: Brij;
   phenol alkyl and PEG ethers;
   surfactants having an amide function:
      copra fatty acid, lauric acid monoethanolamide, etc.
      myristic acid, lauric acid diethanolamide, etc.
      lauric acid mono-isopropanolamine.
         phospholipids such as phosphatidylcholine, phosphatidylserine,
ionic surfactants:
   sulfated derivatives: sodium laurylsulfate and derivatives thereof;
      sulfonated derivatives: sodium dodecylsulfosuccinate and derivatives thereof;
   quaternary ammoniums: cetyltrimethylammonium, laurylpyridinium, distearyldimethylammonium chloride, etc.
amphoterics: copra alkyldimethyl ammonium betain, fatty acid derivatives with a betaine structure, lauryl-α-iminodipropionic acid and derivatives thereof, lauryl-myristyl-α-aminopropionic acid and derivatives thereof, etc.

The amount of these substances, used to promote solubilization or dispersion of active principles may vary from 0.1% to 10% by weight with respect to the total weight of the oil phase.

In the case of certain pasty compositions for topical administration, it is possible to incorporate carriers facilitating the penetration of the active principles. The greater the difficulty of the chondroitin sulfate, being a large molecule, passing the epidermal barrier, the more important these carriers are.

Among the substances facilitating this transdermal passage are camphor, menthol, dimethylsulfoxide and methylal.

The concentrations of these different carriers may range, according to their activity, from 1% to 50% by weight with respect to the total weight of the composition.

Regardless of the type of composition according to the present invention, the protocol for production of the liquid or pasty composition plays an important role in the stability of the end product.

Indeed, the timing of the incorporation of the different ingredients during the production process may or may not determine the appearance of the brown coloring, characterizing the Maillard reaction. The stabilization of these compositions is obtained by chronological solubilization of the glucosamine, then the carboxylic acid, and finally the chondroitin sulfate. It is indeed essential to dissolve the glucosamine first, then to add the acid in the predefined proportions in order to inhibit the reaction between the glucosamine and the chondroitin. The agitation time is dependent on the amounts used and would not be less than 5 minutes for a volume of 100 ml. This mixture can be produced under heat by bringing the solution to 100° C. for the entire agitation time. After cooling, or the time mentioned in order for the reaction to take place under cold conditions, the chondroitin sulfate is added. The mixing time is dependent on the amount used.

The other ingredients intended to obtain a more or less viscous liquid aqueous composition are added to this phase thus obtained. It can be in the form of a suspension or not, in which, in either case, the pH must be adjusted in order to maintain an acid environment of which the values are between 2.0 and 5.0.

An oil phase can be added to the aqueous glucosamine/chondroitin sulfate phase, resulting in more or less viscous emulsions of which the pH must absolutely be adjusted to between 2.0 and 5.0.

Such compositions thus have a degradation rate of less than 10% after 10 months when they are stored at 25° C. and 60% relative humidity.

1. Liquid or pasty compositions based on glucosamine and chondroitin sulfate intended to provide, systemically (orally) or locally (topically), essential elements for the synthesis and constitution of proteoglycans, characterized in that the combination of chondroitin sulfate/glucosamine is stabilized in the end product by the addition of carboxylic acids with a ratio characterizing the number of moles of acid used to block 1 mole of glucosamine ranging from 1:0.05 to 1:1.5 in order to ensure the blocking of the amine function of the glucosamine, resulting in a pH of the medium of between 2.0 and 5.0 and a chemical degradation of the active substances below 10% at 25° C. and 60% relative humidity after 10 months, for doses of between 300 and 2400 mg for chondroitin sulfate and between 500 and 3000 mg for glucosamine.

2. Liquid or pasty compositions according to claim 1, characterized in that the carboxylic acids are aliphatic and aromatic.

3. Liquid or pasty compositions according to claim 1, characterized in that the carboxylic acids a COOH function.

4. Liquid or pasty compositions according to claim 1, characterized in that the carboxylic acids used are acetic acid and derivatives thereof, adipic acid, azelaic acid, butyric acid and derivatives thereof, citramalic acid, citric acid and derivatives thereof, decanoic acid, diglycolic acid, dodecanedioic acid, trans-2-dodecene 1,12-dioic acid, formic acid, fumaric acid, gluconic acid, glutaric acid and derivatives thereof, glycolic acid, glyoxylic acid, hexadecanedioic acid, hexadiene-2,4-dioic acid, hexanoic acid and derivatives thereof, trans-hexene-3-dioic acid, lactic acid, lauric acid, levulinic acid, linoleic acid, maleic acid, malic acid, malonic acid, mellitic acid and derivatives thereof, methyl-3-crotonic acid, methyl-3-glutyric acid, mucic acid, myristic acid, octanoic acid, oenanthic acid, oleic acid, oxalic acid and derivatives thereof, palmitic acid, pelargonic acid, pentadecanoic acid, trans-pentenoic acid, pentynoic acid, pimelic acid, pivalic acid, propionic acid, propyl-2-valerianic acid, pyruvic acid, sebacic acid, sorbic acid, stearic acid, suberic acid, succinic acid and derivatives thereof, tartric acid, tetradecanedioic acid, tiglic acid, tridecanoic acid, undecylenic acid and valeric acid and derivatives thereof.

5. Liquid or pasty compositions according to claim 1, characterized in that the pH of the medium is stabilized at between 2.0 and 5.0 by the addition of buffer solutions.

6. Liquid or pasty compositions according to claim 1, characterized in that they contain sweeteners and/or flavor enhancers and/or flavors, and/or coloring agents and/or preservatives and/or suspension agents, and/or surfactants and/or organic carriers and/or substances facilitating transdermal passage.

7. Liquid or pasty compositions according to claim 1, characterized in that they contain other active substances such as anti-inflammatories, medicinal plants having an action in the field of joints, vitamins, minerals and organic sulfur derivatives.

8. Liquid or pasty compositions according to claim 1, characterized in that the stabilization of these compositions is obtained by chronological solubilization of the glucosamine, then the carboxylic acid and finally the chondroitin sulfate.

9. Liquid or pasty compositions based on glucosamine and chondroitin sulfate according to claim 1, characterized in that they are intended to treat cartilage degradation.

10. Liquid or pasty compositions based on glucosamine and chondroitin sulfate according to claim 1, characterized in that the compositions, in which the concentration (by weight with respect to the total weight) of glucosamine is between 0.10% and 15% and that of chondroitin sulfate is between 0.06% and 12% are presented as a single dose administered once per day.

11. Liquid or pasty compositions based on glucosamine and chondroitin sulfate according to claim 1, characterized in that the compositions, in which the concentration (by weight with respect to the total weight) of glucosamine is between 2.0% and 20% and that of chondroitin sulfate is between 1% and 15% are presented as multi-doses administered a plurality of times over several days.
